(19) United States
(12) Patent Application Publication
(10) Pub. No.: US 2024/0307342 A1
(43) Pub. Date: Sep. 19, 2024

(54) Treatment of the adverse cardiovascular effects of cannabinoids

(71) Applicant: GreenStone Biosciences, Inc., Palo Alto, CA (US)

(72) Inventors: Joseph C Wu, Palo Alto, CA (US); Jade Chao, Palo Alto, CA (US); Mark Chandy, London (CA); Paul Pang, Redwood City, CA (US)

(73) Assignee: GreenStone Biosciences, Inc., Palo Alto, CA (US)

(21) Appl. No.: 18/139,917

(22) Filed: Apr. 26, 2023

Related U.S. Application Data
(60) Provisional application No. 63/336,294, filed on Apr. 28, 2022.

(51) Int. Cl. A61K 31/352; A61K 9/10; A61K 47/30; A61P 9/00

(52) U.S. Cl. CPC A61K 31/352; A61K 9/10; A61K 47/30; A61P 9/00

(57) ABSTRACT

Treatment of the adverse cardiovascular effects of cannabinoids in a subject with genistein or a genistein derivative.

### CROSS-REFERENCE TO RELATED APPLICATION

This application claims the benefit under 35 USC 119(e) of Application No. 63/336,294, filed 28 Apr. 2022, the entire content of which is incorporated into this application by reference.

### BACKGROUND OF THE INVENTION

### Field of the Invention

This invention relates to the treatment of the adverse cardiovascular effects of cannabinoids with genistein or a genistein derivative.

### Description of the Related Art

### Cannabinoids

Medical marijuana or cannabis has been used for several thousand years. Cannabis and its medicinal preparations from Cannabis indica and C. sativa have been used for treating nausea, inflammation, vomiting, and pain. The legalization of marijuana in many states resulted in an increase in its use, both recreationally and for medicinal purposes. The most important component of the cannabis plant is a group of plant chemicals called cannabinoids or phytocannabinoids, which give the cannabis plant medical and recreational properties. Cannabinoids, either as cannabis plant or parts thereof, such as buds, or as resins, or extracts (e.g., “hash oil”) are ingested recreationally various means, typically by smoking, “vaping” of extracts, or oral consumption of cannabis plant or parts, or extracts, in foodstuffs, such as confections; and ingested medicinally typically as dosage forms (e.g. capsules, sprays) containing known quantities of pure cannabinoids. The Institute of Medicine recommended testing alternative cannabinoid delivery systems to smoking, and conducting clinical trials to assess efficacy of synthetic and plant-derived cannabinoids for the treatment of spasticity, movement disorders, glaucoma, and other indications. As discussed later, cannabinoid preparations containing natural or synthetic cannabinoids are available as prescription drugs in some countries.

It has been demonstrated that cannabinoids are a class of diverse chemical compounds that act on cannabinoid receptors in cells that alter neurotransmitter release in the brain. Over a hundred cannabinoids have been identified from cannabis, of which the four most prominent are Δ⁹-tetrahydrocannabinol (Δ⁹-THC, THC, (6aR,10aR)-6,6,9-trimethyl-3-pentyl-6a,7,8,10a-tetrahydro-6H-benzo[c]chromen-1-ol), cannabidiol (CBD, 2-[(1R,6R)-6-isopropenyl-3-methyl-2-cyclohexen-1-yl]-5-pentyl-1,3-benzenediol), Δ⁸-tetrahydrocannabinol (Δ⁸-THC, (6aR)-6,6,9-trimethyl-3-pentyl-6a,7,10,10a-tetrahydro-6H-benzo[c]chromen-1-ol), and cannabinol (CBN, 6,6,9-trimethyl-3-pentyl-6H-benzo[c]chromen-1-ol); of which Δ⁹-THC and CBD are the most extensively studied. Δ⁹-THC and Δ⁸-THC are both psychoactive, while CBD is reported to possess anxiolytic, antipsychotic, antiemetic, and anti-inflammatory properties, without exhibiting the psychoactive effects of THC. Δ⁹-THC and CBD are biosynthesized as Δ⁹-tetrahydrocannabinolic acid and cannabidiolic acid from the common precursor olivetol.

Both THC and CBD exert their effects by interacting with the G protein-coupled receptors (GPCRs) cannabinoid receptor 1 (CB1) and cannabinoid receptor 2 (CB2), with varying affinities. While CB1 receptors are expressed in large quantities in the brain and regions in the central nervous system, and in lower amounts in peripheral tissues; the less studied CB2 receptors have been identified to be localized to immune cells, tonsils, and the spleen. The CB1 receptors have been identified to play significant roles in pain perception, memory, motor regulation, appetite, mood and sleep, and regulate the psychoactive effects of THC and related cannabinoids; whereas the CB2 receptors have been linked with anti-inflammation, pain reduction and reducing tissue damage. Physiologically, upon activation by the endocannabinoids like anandamide and 2-arachidonylglycerol, which are short lived, CB1 and CB2 trigger a downstream cascade of events that mediate homeostasis and healthy functioning. In contrast, the cannabinoids THC and CBD that directly or indirectly interact with CB1 and CB2 with varying affinities modulate the activities of these receptors for prolonged durations.

It is well known that cannabinoids, especially CBD and THC, have many medicinal benefits. The conditions include post-traumatic stress disorder, neuropathic and chronic pain, pulmonary hypertension, systemic hypertension, insomnia, nausea, inflammation, arthritis, migraines, cancer, Crohn's disease, fibromyalgia, Alzheimer's disease, multiple sclerosis, glaucoma, attention deficit hyperactivity disorder (ADHD), sleep apnea and appetite loss. CBD's subtle effects are primarily felt in pain, inflammation, and anxiety relief, as well as other medicinal benefits. CBD also does not have any adverse side effects that may occur with consumption of THC. Unlike THC, CBD also does not cause a high. CBD also appears to counteract the sleep-inducing effects of THC, which may explain why some strains of cannabis are known to increase alertness.

The U.S. Food and Drug Administration (FDA) has approved two synthetic cannabinoids for treating chemotherapy-induced nausea and vomiting and HIV-associated anorexia: dronabinol, synthetic Δ⁹-THC, as MARINOL capsules; and nabilone, 1-hydroxy-6,6-dimethyl-3-(2-methyl-2-octanyl)-6,6a,7,8,10,10a-hexahydro-9H-benzo[c]chromen-9-one, a THC analog, as CESAMET capsules. Synthetic cannabinoids are also associated with adverse cardiovascular effects. CBD is also approved, as EPIDIOLEX oral solution, for treating seizures associated with Lennox-Gastaut syndrome, Dravet syndrome, or tuberous sclerosis complex in patients 1 year of age and older. Nabiximols is a cannabis extract whose principal active components are THC and CBD. It is approved in various countries, although not the US, as SATIVEX oral spray, for treating neuropathic pain, spasticity, overactive bladder, and other symptoms of multiple sclerosis, with each spray delivering a dose of 2.7 mg THC and 2.5 mg CBD.

DeFillipis et al., “Cocaine and marijuana use among young adults with myocardial infarction”, J. Am. Coll. Cardiol., 71, 2540-2551 (2018) have shown in a cohort of 2,097 patients that marijuana is associated with MI in young adults when controlling for other demographic factors. Previous studies have shown that CB1 activation is proatherogenic by promoting inflammation and oxidative stress that cause endothelial dysfunction and atherosclerosis, whereas CB2 activation is anti-atherogenic. Retrospective studies indicate that use of marijuana increases the risk of cardiovascular disease (CVD) including myocardial infarction (MI), angina and arrhythmias. Marijuana exposure induced endothelial dysfunction, atherosclerosis, cardiomyopathy and metabolic dysfunction in an animal model. The “adverse cardiovascular effects of a cannabinoid” thus include the effects mentioned in this paragraph.

CB1 antagonists have been shown to be anti-atherogenic, and CB2 agonists are in development for vascular disorder. CBD has low affinity for the cannabinoid CB1 and CB2 receptors, although it can act as an antagonist of CB1/CB2 agonists despite this low affinity. CBD may act as an inverse agonist of GPR3, GPR6 and GPR12. CBD has been shown to act as a serotonin 5-HT_{1A} receptor partial agonist and this action may be involved in its antidepressant, anxiolytic, and neuroprotective effects. It is also an allosteric modulator of the μ- and δ-opioid receptors. The pharmacological effects of CBD may involve PPARγ agonism and intracellular calcium release.

Because both recreational and medicinal users of cannabinoids, such as recreational smokers of cannabis and medicinal users of cannabinoid-containing drugs such as dronabinol and nabilone, are at risk for the adverse cardiovascular effects of these cannabinoids, it is desirable to find a treatment for these adverse cardiovascular effects.

### Genistein

Genistein, 5,7-dihydroxy-3-(4-hydroxyphenyl)-4H-1-benzopyran-4-one; 4′,5,7-trihydroxyisoflavone; sometimes incorrectly 4′,5,7-trihydroxyflavone, is a phytoestrogen belonging to the class of soy isoflavones. It was first isolated in 1899 from the dyer's broom, Genista tinctoria; its structure was established in 1926; and it was chemically synthesized in 1928 (Walter, “Genistin (an Isoflavone Glucoside) and its Aglucone, Genistein, from Soybeans”, J. Am. Chem. Soc., 63(12), 3273-3276 (1941)). Of the soy isoflavones, genistein ranks first in the number of experimental and clinical studies performed. Genistein itself is found in low concentrations in soybeans, but at high concentrations in fermented soy-derived foods such as miso and natto. It is also widely distributed in leguminous plant foods as well as in seeds, fruits, and vegetables such as alfalfa and clover sprouts, broccoli, cauliflower, sunflower, barley meal, caraway, and clover seeds. It is widely available in the US as a nutritional supplement, frequently in combination with other isoflavones such as genistin (genistein 7-O-β-D-glucoside) and daidzein.

Many derivatives of genistein have been synthesized: Spagnuolo et al., “Genistein and Cancer: Current Status, Challenges, and Future Directions”, Adv. Nutr., 6, 408-419 (2015) say: “On this basis and because of the known beneficial biological effects of genistein, chemists have been so far encouraged to synthesize many derivatives of this compound, with improved pharmacologic profile. For instance, FA-esterified, 6-carboxymethyl, nitroxy, 7-O-heterocycle, 7-O-β-D-glucoside and 7-O-β-D-glucuronic acid, halogenated, deoxybenzoin, benzylated, hydroxylated, esterified, benzosulfonate, dimethylaminomethyl, phenoxyalkylcarboxylic acid, glycoconjugate, and alkylbenzylamine derivatives of genistein have been reported to date”, citing 15 references. As a few examples of the genistein derivatization literature, Grynkiewicz et al., “X-ray and 13C CP MAS investigations of structure of two genistein derivatives”, J. Mol. Struct., 694, 121-129 (2004) report the synthesis of the 7-O-(t-butyldimethylsilyl) ether and 7-O-(tetrabutylammonium) salt of genistein, useful as intermediates to other 7-substituted genistein derivatives. Rusin et al., “Synthetic conjugates of genistein affecting proliferation and mitosis of cancer cells”, Bioorg. Med. Chem., 19, 295-305 (2011), report the syntheses of 7-O-ω-hydroxyalkylgenisteins and conjugates. Szeja et al., “Selective alkylation of genistein and daidzein”, Chem. Biol. Interface, 3, 95-106 (2013), describe regioselective 7-O- and 4-O-alkylations of genistein, including the preparation of dialkylated derivatives; while Byczek et al., “Genistein Derivatives Regioisomerically Substituted at 7-O- and 4′-O- Have Different Effect on the Cell Cycle”, J. Chem., 2013, 191563, describe further regioisomerically substituted genistein derivatives. Castro et al., “Synthesis of Lipophilic Genistein Derivatives and Their Regulation of IL-12 and TNF-α in Activated J774A.1 Cells”, Chem. Biol. Drug Des., 79, 347-352 (2012) report the synthesis of both acylated and alkylated derivatives of genistein, including general procedures for the synthesis of these classes of genistein derivatives.

Crystalline genistein monohydrate and crystalline genistein salts, such as the sodium, potassium, calcium, magnesium, L-lysine, and N-methylglucamine salts, are known in, for example, PCT International Publication No. WO 2010/068861 A1, “Crystalline forms of genistein”, Axcentua Pharmaceuticals AB.

Genistein influences multiple biochemical functions in living cells: it is a full agonist of estrogen receptor β (ERβ; EC₅₀=7.6 nM), and, to about a 20-fold lesser extent, a full or partial agonist of ERα; an agonist of the G protein-coupled estrogen receptor (affinity=133 nM); and activator of peroxisome proliferator-activated receptors; and an inhibitor of several tyrosine kinases, the EFGR kinase and the BCR-ABL protein specific tyrosine kinase. Genistein is reported as an agonist of human CB1, with IC₅₀=0.15 μM and Ki=0.14 μM, and an agonist of human CB2, with IC₅₀ and Ki both>50 μM. Genistein is a well-studied anticancer agent, reportedly active through multiple mechanisms, including as an angiogenesis inhibitor, a DNA topoisomerase II inhibitor, and as a tyrosine kinase inhibitor. Reports on the effect of genistein on appetite are mixed.

While genistein is widely available in the United States as a dietary supplement, genistein is known to have low solubility and therefore low bioavailability, even though it has high cell wall permeability; and various attempts have been made to increase bioavailability of genistein both by chemical modification (sec the first paragraph of this subsection) and by physical means, such as by milling into submicron particles and administration as a suspension (sec PCT International Publications No. WO 2012/068140 A1, “Nanoparticle isoflavone compositions & methods of making and using the same”, and WO 2015/081018 A1, “Suspension compositions of physiologically active phenolic compounds & methods of making and using the same”, both to Humanetics Corporation). Humanetics' suspension formulation, BIO 300, which contains 325 mg/mL of wet-milled genistein with an average particle size of 200 nm, has been tested in both animal and Phase 1 clinical studies in humans.

Genistein is known to protect against radiation-induced fibrosis in animal models, and several articles describe the protective effects of the Humanetics BIO 300 formulations in murine models of radiation-induced fibrosis. See, for example, Jackson et al., “BIO 300, a nanosuspension of genistein, mitigates pneumonitis/fibrosis following high-dose radiation exposure in the C57L/J murine model”, Br. J. Pharmacol., 174(24), 4738-4750 (2017), and Singh et al., “A novel formulation of BIO 300 confers prophylactic radioprotection from acute radiation syndrome in mice”, Int. J. Radiat. Biol., 98(5), 958-967 (2022). The latter article, in the “Test and Control Items” section, gives details of the formulation of the BIO 300 injectable, oral suspension, and powder formulations.

The disclosures of the documents referred to in this application are incorporated into this application by reference.

### SUMMARY OF THE INVENTION

This invention is methods of treating the adverse cardiovascular effects of a cannabinoid in a subject by administration of genistein or a genistein derivative.

In another aspect, this invention includes pharmaceutical compositions comprising a cannabinoid and genistein or a genistein derivative.

Because genistein has been shown to treat the adverse cardiovascular effects of Δ⁹-THC in various models, genistein and genistein derivatives are expected to treat the adverse cardiovascular effects of cannabinoids in humans.

Preferred embodiments of this invention are characterized by the specification and by the features of Claims 1 to 15 of this application as filed.

### DETAILED DESCRIPTION OF THE INVENTION

### Defintions

“Cannabinoids” are described in the section entitled “Cannabinoids” in the DESCRIPTION OF THE RELATED ART; and include THC, nabilone, and CBD; in particular, those cannabinoids which are CB1 agonists, such as THC. Unless the context requires otherwise, the term “a cannabinoid” includes more than one cannabinoid (“cannabinoids”), and, for example, includes cannabis plants and parts thereof, such as the buds, cannabis resins, cannabis extracts such as “hash oil” and the like, and cannabis or cannabis extract-containing foodstuffs, as long as they contain one or more cannabinoids, and also cannabinoid-containing prescription drugs.

The “adverse cardiovascular effects of a cannabinoid” are described in the sixth paragraph of the section entitled “Cannabinoids” in the section entitled “Cannabinoids” in the DESCRIPTION OF THE RELATED ART. In particular, these adverse cardiovascular effects are the adverse cardiovascular effects associated with the CB1 agonism of the cannabinoids. In particular aspects, the adverse cardiovascular effect is one or more effects selected from the group consisting of myocardial infarction (MI), angina, arrhythmias, endothelial dysfunction, atherosclerosis, cardiomyopathy and metabolic dysfunction.

“Genistein” is described in the section entitled “Genistein” in the DESCRIPTION OF THE

### RELATED ART

“Genistein or a genistein derivative” means a compound of formula I:

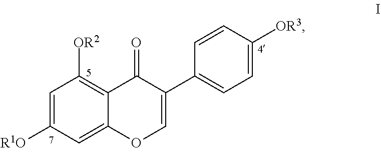

or a pharmaceutically acceptable salt thereof,
where each of R¹, R² and R³ independently is selected from the group consisting of H, (HO)₂P(O)—, HOS(O)₂—, H₂NS(O)₂—, H(OCH₂CH₂)₁₋₁₀—, (CH₃)₂NCH₂CH₂—, morpholino, (C₁₋₆ alkyl)C(O)—[where the C₁₋₆ alkyl is optionally substituted with 1 or 2 substituents selected from the group consisting of halo, hydroxy, C₁₋₄ alkoxy, amino, C₁₋₄ alkylamino, cyano, and acetoxy], and an acyl group of an amino acid or hydroxy acid. “Acyl group of an amino acid” means that portion of an amino acid that is derived from the amino acid by removal of a hydroxy group from a carboxyl group of the amino acid. “Acyl group of a hydroxy acid” means that portion of a hydroxy acid that is derived from the hydroxy acid by removal of a hydroxy group from a carboxyl group of the hydroxy acid. “A genistein derivative” means a compound of formula I as defined above where at least one of R¹, R² and R³ is not H. “Genistein or a genistein derivative” includes these compounds in both crystalline and amorphous forms, and in micronized form, including in formulations such as in the Humanetics Corporation BIO 300 suspension. Unless the context requires otherwise, the term “genistein” means “genistein or a genistein derivative” as defined in this paragraph.

Salts (for example, pharmaceutically acceptable salts) of genistein or a genistein derivative are included in this invention and are useful in the methods described in this application. These salts are preferably formed with pharmaceutically acceptable acids or bases, as appropriate. See, for example, “Handbook of Pharmaceutically Acceptable Salts”, Stahl and Wermuth, eds., Verlag Helvetica Chimica Acta, Zurich, Switzerland, for an extensive discussion of pharmaceutical salts, their selection, preparation, and use. Unless the context requires otherwise, any reference to genistein or a genistein derivative is a reference both to the compound and to its salts. Genistein can exist as the non-salt compound, but salts with alkali metals and nitrogenous bases (e.g., tromethamine) are known, for example, PCT Publication No. WO 2010/068861, referred to previously, discloses both alkali metal and nitrogenous base salts.

A “therapeutically effective amount” of genistein or a genistein derivative means that amount of each which, when the genistein derivative is administered to a human for treating the adverse cardiovascular effects of a cannabinoid, is sufficient to effect treatment for the adverse cardiovascular effects of a cannabinoid.

“Treating” or “treatment” of the adverse cardiovascular effects of a cannabinoid in a human includes one or more of:
(1) preventing or reducing the risk of developing an adverse cardiovascular effect of a cannabinoid, i.e., causing the clinical symptoms of the adverse cardiovascular effect not to develop in a subject who may be predisposed to them but who does not yet experience or display symptoms (i.e., prophylaxis);
(2) inhibiting the adverse cardiovascular effect of a cannabinoid, i.e., arresting or reducing the development of the adverse cardiovascular effect or its clinical symptoms; and
(3) relieving the adverse cardiovascular effect of a cannabinoid, i.e., causing regression, reversal, or amelioration of the adverse cardiovascular effect or reducing the number, frequency, duration or severity of its clinical symptoms.
   “Treatment” does not necessarily imply “cure” or complete treatment, e.g., treatment of all clinical symptoms of the adverse cardiovascular effect of a cannabinoid, though “treatment” may include “cure”. Rather, “treatment” implies the provision of clinical benefit by the administration of the genistein derivative when compared to non-administration of genistein or a genistein derivative; and treatment may also be assessed by improvement in biological markers of the adverse cardiovascular effect of a cannabinoid being treated.
   The therapeutically effective amount for a particular subject varies depending upon the health and physical condition of the subject to be treated, the extent of the adverse cardiovascular effect of a cannabinoid, the assessment of the medical situation, and other relevant factors. It is expected that the therapeutically effective amount will fall in a relatively broad range that can be determined through routine trial.

“Comprising” or “containing” and their grammatical variants are words of inclusion and not of limitation and mean to specify the presence of stated components, groups, steps, and the like but not to exclude the presence or addition of other components, groups, steps, and the like. Thus “comprising” does not mean “consisting of”, “consisting substantially of”, or “consisting only of”; and, for example, a formulation “comprising” a compound must contain that compound but also may contain other active ingredients and/or excipients.

Subjects Suitable for Treatment by this Invention

Suitable subjects for treatment by this invention are humans suffering from an adverse cardiovascular effect of a cannabinoid.

Genistein Derivatives and their Synthesis

Genistein derivatives of particular interest are those mono-substituted at the 4′ or 7 positions, or di-substituted at the 4′ and 7 positions, i.e., those compounds in which R² is hydrogen and R¹ and/or R³ are not hydrogen; though tri-substituted compounds are also of interest. Where any one or more of R¹, R², and R³, are an “acyl group of an amino acid”, the amino acid is particularly alanine, aspartic acid, cysteine, glutamic acid, glycine, valine, isoleucine, leucine, methionine, phenylalanine, proline, serine, threonine, tyrosine or tryptophan. Where any one or more of R¹, R², and R³, are an “acyl group of a hydroxy acid”, the hydroxy acid is particularly an ω-hydroxy acid, such as δ-hydroxypentanoic acid and the like.

Genistein derivatives of particular interest are compounds of formulae Ia to I1:

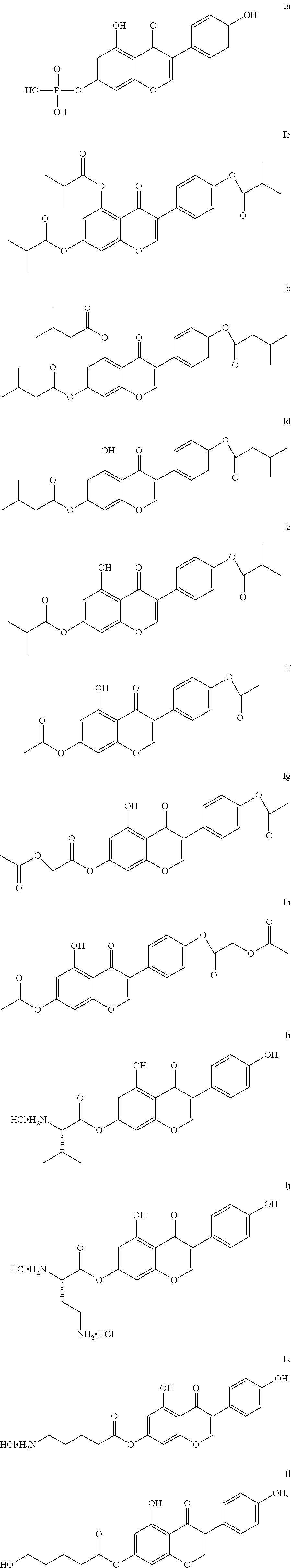

and the pharmaceutically acceptable salts thereof.

The genistein derivatives of this invention may be prepared by conventional methods of organic synthesis well-known to those of ordinary skill in the art, having regard to that knowledge and the known methods of synthesis of genistein derivatives as exemplified in the documents referred to in the second and third paragraphs of the section entitled “Genistein” in the DESCRIPTION OF THE RELATED ART, and others.

For example, as described in Szeja et al., 4′-O-alkylation may be performed by treating genistein with an excess of strong base such as potassium tert-butoxide in an aprotic solvent such as dimethylformamide (DMF), then adding the relevant alkylating agent such as an alkyl halide or sulfonate. 7-O-Alkylation may conveniently be performed by preparing the tetrabutylammonium salt of genistein as described in Grynkiewicz et al., reacting a methanol suspension of genistein with a slight excess of an aqueous solution of tetrabutylammonium hydroxide, and then treating that salt with the relevant alkylating agent in an aprotic solvent. Di-alkylation may conveniently be performed by 4′-O-alkylation of a 7-O-alkylated genistein derivative.

Acylations of genistein, or of alkylated derivatives of genistein, may conveniently be performed by conventional methods, such as the reaction of genistein with the relevant acid in the presence of coupling reagents such as dicyclohexylcarbodiimide and hydroxybenzotriazole in aprotic solvents such as dichloromethane. The acylated genistein may conveniently be isolated as a salt, for example the hydrochloride salt, by treatment of the reaction mixture with hydrochloric acid. Other methods include the use of acyl halides or acid anhydrides and an organic base such as triethylamine, pyridine, or dimethylaminopyridine, again in an aprotic solvent such as dichloromethane. Genistein phosphonate derivatives may be performed under the standard procedure using genistein and phosphonyl halide, such as phosphonyl chloride, under similar conditions; as may genistein sulfonates and sulfonamides using the relevant sulfonyl halide.

In each case, confirmation of the identity of the product may be obtained using conventional methods of analysis, such as nuclear magnetic resonance.

### Formulation and Administration

The genistein or a genistein derivative may be administered by any route suitable to the subject being treated and the nature of the subject's condition. Routes of administration include oral administration (generally preferred, if available); administration by injection, including intravenous, intraperitoneal, intramuscular, and subcutaneous injection; by transmucosal (e.g., intranasal, buccal, sublingual, rectal, or vaginal) or transdermal (topical) delivery; and the like. Formulations may be oral formulations (e.g., tablets, capsules, or oral solutions or suspensions); injectable formulations (e.g., solutions); and formulations designed to administer the drug across mucosal membranes or transdermally. Suitable formulations for each of these methods of administration may be found, for example, in “Remington: The Science and Practice of Pharmacy”, 20th ed., Gennaro, ed., Lippincott Williams & Wilkins, Philadelphia, Pa., U.S.A. Because both genistein and the statins are generally orally available, typical formulations will be oral, and typical dosage forms will be tablets or capsules for oral administration. Intravenous formulations may be particularly applicable for administration to acutely ill subjects, such as those subjects who may be hospitalized for treatment.

Depending on the intended mode of administration, the pharmaceutical compositions may be in the form of solid, semi-solid or liquid dosage forms, preferably in unit dosage form suitable for single administration of a precise dosage. In addition to an effective amount of the tyrosine kinase inhibitor and/or the statin, the compositions may contain suitable pharmaceutically-acceptable excipients, including adjuvants which facilitate processing of the active compounds into preparations which can be used pharmaceutically. “Pharmaceutically acceptable excipient” refers to an excipient or mixture of excipients which does not interfere with the effectiveness of the biological activity of the active compound(s) and which is not toxic or otherwise undesirable to the subject to which it is administered.

For solid compositions, conventional pharmaceutically acceptable excipients include, for example, pharmaceutical grades of mannitol, lactose, starch, magnesium stearate, sodium saccharin, talc, cellulose, glucose, sucrose, magnesium carbonate, and the like. Liquid pharmacologically administrable compositions can, for example, be prepared by dissolving, dispersing, etc., an active compound as described herein and optional pharmaceutical adjuvants in water or an aqueous excipient, such as, for example, water, saline, aqueous dextrose, and the like, to form a solution or suspension. If desired, the pharmaceutical composition to be administered may also contain minor amounts of nontoxic auxiliary excipients such as wetting or emulsifying agents, pH buffering agents and the like, for example, sodium acetate, sorbitan monolaurate, triethanolamine sodium acetate, triethanolamine oleate, etc. When liquid suspensions are used, the active agent may be combined with emulsifying and suspending excipients. If desired, flavoring, coloring and/or sweetening agents may be added as well. Other optional excipients for incorporation into an oral formulation include preservatives, suspending agents, thickening agents, and the like.

Nanoparticulate formulations of genistein or a genistein derivative, such as the submicron particle suspensions of genistein developed by Humanetics Corporation and described in PCT International Publications No. WO 2012/068140 A1, “Nanoparticle isoflavone compositions & methods of making and using the same”, and WO 2015/081018 A1, “Suspension compositions of physiologically active phenolic compounds & methods of making and using the same”, both to Humanetics Corporation, may be of particular interest.

In one embodiment, the nanoparticles of genistein or a genistein derivative in the formulation may have a particle size that is less than 250 nm (0.25 microns), as measured using a dynamic light scattering (DLS) detector, for example, and may have a median (average) particle size of less than about 200 nm, less than 150 nm, less than 100 nm, less than about 90 nm, less than about 80 nm, less than about 70 nm or less than about 60 nm. In a further example, the particle may have a median particle size of less than about 50 nm, less than about 40 nm or less than about 30 nm. In another embodiment, the average particle size is from about 7 nm to about 90 nm. In another embodiment, the average particle size is from about 5 nm to about 70 nm, from about 10 nm to about 50 nm, from about 10 nm to about 30 nm, or from about 7 nm to about 10 nm. In a particular example, the particle may have a median particle size between about 30 nm and about 10 nm (e.g., about 25 nm).

In another embodiment, the nanoparticle formulation of genistein or a genistein derivative (i.e., the nanoparticulate formulation) may be a suspension formulation that include the nanoparticulate of genistein or a genistein derivative suspended in a suspension medium formed of one or more carriers, excipients, and/or diluents. Pharmaceutically acceptable carriers, excipients and diluents suited for therapeutic use are well known in the pharmaceutical art, and are described, for example, in Remington's Pharmaceutical Sciences, Mack Publishing Co. (A. R. Gennaro (Ed.) (1985). In another embodiment, the nanoparticle formulation of genistein or a genistein derivative may be a suspension comprising the nanoparticulate suspended within a suspension medium including a water soluble polymer and a nonionic surfactant. The pharmaceutical compositions may be formulated to be suitable for both oral and parenteral administration. As disclosed herein, depending on the particular formulation employed, a “suspension” of the nanoparticulate may also include a measurable amount of the genistein or a genistein derivative dissolved within the suspension medium.

The above nanoparticulate formulation may be prepared using known methods for preparing nano-sized particles. For example, the genistein or a genistein derivative may be nano-milled according to milling techniques known in the art, including wet bead milling using an agitator bead mill in a horizontal grinding container for continuous dispersion and fine wet grinding. Or the milling may employ a bead mill such as a DYNO-mill (CB Mills, Gurnee, Ill.) to provide a dispersion. See, for example, Loh et al., “Overview of milling techniques for improving the solubility of poorly water-soluble drugs”, Asian J. Pharm. Sci., 10, 255-274 (2015).

In one embodiment, the nanoparticulate formulation may be prepared by adding the genistein or a genistein derivative that is suspended in a pharmaceutically acceptable suspension medium into a bead mill, and milled in a manner that results in a pharmaceutical formulation as characterized by nanoparticulate formulation within the pharmaceutically acceptable suspension medium. The formulation may be nano-milled by recirculating the volume of the suspension through the bead mill, followed by one or more passes through a bead mill to prepare a pharmaceutical composition exhibiting the desired particle size distribution. The particle size of the suspended nanoparticle within a pharmaceutical composition can be controlled by adjusting the parameters of the bead mill and the grinding conditions, including the bead size, bead load/suspension weight ratio, suspension composition, agitation rate and milling time.

Alternatively, the nanoparticulate formulation may be prepared by employing one or more known wet milling techniques, super-critical or compressed fluid techniques, hot or high-pressure homogenization, emulsification techniques, evaporative precipitation, antisolvent precipitation, microprecipitation, cryogenic techniques, complexation techniques, ultrasonication techniques, or solid dispersion techniques as known in the art. Spray drying and lyophilization may be used post-processing to isolate nanoparticles that may be prepared from an aqueous or solvent dispersion technique.

In one embodiment, the nanoparticulate formulation may comprise the nanoparticulate genistein or genistein derivative with a D (0.50) of less than 1.0 μm, less than 0.8 μm, less than 0.7 μm, less than 0.5 μm, or less than 0.3 μm.

The nanoparticular formulation may include one or more surfactants or solubilizing agents that may be selected from Poloxamer 188, Polysorbate 80, Polysorbate 20, Vit E-TPGS (TPGS), TPGS-750-M, TPGS-1000, Solutol HS 15, PEG-40 Hydrogenated castor oil (Cremophor RH40, Cremophor RH60), PEG-35 Castor oil (Cremophor EL), PEG-8-glyceryl caprylate/caprate (Labrasol), PEG-32-glyceryl laurate (Gelucire 44/14), PEG-32-glyceryl palmitostearate (Gelucire 50/13); Polysorbate 85, Polyglyceryl-6-dioleate (Caprol MPGO), mixtures of high and low HLB emulsifiers; Sorbitan monooleate (Span 80), Capmul MCM, Maisine 35-1, glyceryl monooleate, glyceryl monolinoleate, PEG-6-glyceryl oleate (Labrafil M 1944 CS), PEG-6-glyceryl linoleate (Labrafil M 2125 CS), oleic acid, linoleic acid, propylene glycol monocaprylate (e.g. Capmul PG-8 or Capryol 90), propylene glycol monolaurate (e.g., Capmul PG-12 or Lauroglycol 90), polyglyceryl-3 dioleate (Plurol Oleique CC497), where the solubilizing agent maybe present in the formulation in about 0.01% to about 10% by weight (w/w), or about 0.2% to about 1% (w/w).

The nanoparticular formulation may include one or more water soluble polymers. Suitable water soluble polymers include vegetable gums, such as alginates, pectin, guar gum and xanthan gum, modified starches, polyvinyl pyrrolidone (PVP), hydroxypropyl methylcellulose (HPMC), Povidone K17, methylcellulose and other cellulose derivatives, such as sodium carboxymethylcellulose and hydroxypropylcellulose.

Typically, a pharmaceutical composition of genistein a genistein derivative is packaged in a container with a label, or instructions, or both, indicating use of the pharmaceutical composition in the treatment of the adverse cardiovascular effect of a cannabinoid.

A suitable amount of a genistein derivative for oral dosing (subjects with of the adverse cardiovascular effect of a cannabinoid may well be taking other therapies in addition to the genistein or genistein derivative discussed in this application) is expected to be 1-5 mg/Kg/day. That is, a suitable amount of genistein or a genistein derivative for oral dosing is expected to be similar to the amounts employed in current clinical practice for treatment of prostate cancer when used alone.

A person of ordinary skill in the art of the treatment of the adverse cardiovascular effect of a cannabinoid will be able to ascertain a therapeutically effective amount of the genistein or genistein derivative for a particular subject and nature and extent of the adverse cardiovascular effect, to achieve a therapeutically effective amount without undue experimentation and in reliance upon personal knowledge and the disclosure of this application.

### SYNTHESES

### Synthesis 1. Synthesis of [4-[5,7-bis(2-methylpropanoyloxy)-4-oxo-chromen-3-yl]phenyl]2-methylpropanoate (Ib)

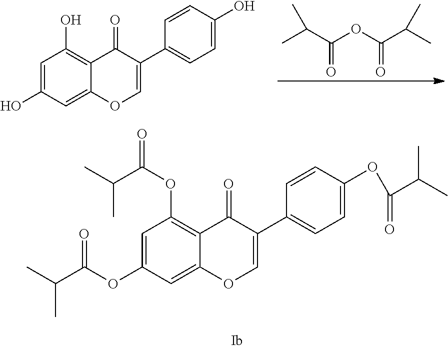

A round bottom flask was charged with 5,7-dihydroxy-3-(4-hydroxyphenyl)chromen-4-one (500 mg, 1.85 mmol), 2-methylpropanoyl 2-methylpropanoate (878 mg, 5.55 mmol), and DMAP (677 mg, 5.55 mmol) in DCM (10 mL). The reaction mixture was stirred at room temperature overnight. Upon completion, the reaction mixture was concentrated under reduced pressure. Chromatography (SiO₂; 0-100% DCM/hexanes) provided the title product as a white solid (0.18 g; 20%).

¹H NMR (500 MHz, CDCl₃) δ 7.88 (d, J=1.7 Hz, 1H), 7.53-7.45 (m, 2H), 7.22 (t, J=2.0 Hz, 1H), 7.12 (dd, J=8.6, 1.8 Hz, 2H), 6.83 (t, J=2.0 Hz, 1H), 3.05-2.94 (m, 1H), 2.82 (ddtd, J=14.0, 8.9, 7.0, 1.7 Hz, 2H), 1.39-1.30 (m, 18H).

### Synthesis 2. Synthesis of [4-[5-hydroxy-7-(2-methylpropanoyloxy)-4-oxo-chromen-3-yl]phenyl]2-methylpropanoate (Ie)

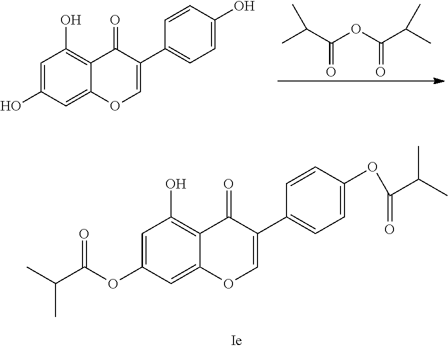 [SMILES: O=c1c(-c2ccc(O)cc2)coc2cc(O)cc(O)c12 ; CC(C)C(=O)OC(=O)C(C)C ; CC(C)C(=O)Oc1ccc(-c2coc3cc(OC(=O)C(C)C)cc(O)c3c2=O)cc1]

A round bottom flask was charged with 5,7-dihydroxy-3-(4-hydroxyphenyl)chromen-4-one (500 mg, 1.85 mmol), 2-methylpropanoyl 2-methylpropanoate (585 mg, 3.7 mmol), and DMAP (248 mg, 2.03 mmol) in DCM (10 mL). Pyridine (0.44 mL, 5.55 mmol) was added and the reaction mixture was stirred at room temperature overnight. Upon completion, the reaction mixture was concentrated under reduced pressure. Chromatography (SiO₂; 0-25% EtOAc in hexanes) provided the title product as a white solid (0.11 g; 14%).

¹H NMR (500 MHz, CDCl₃) δ 12.74 (s, 1H), 7.97 (s, 1H), 7.57-7.53 (m, 2H), 7.19-7.16 (m, 2H), 6.76 (d, J=2.1 Hz, 1H), 6.58 (d, J=2.1 Hz, 1H), 2.85-2.80 (m, 2H), 1.34 (d, J=7.0 Hz, 12H).

### Synthesis 3. Synthesis of [4-[5,7-dihydroxy-4-oxo-chromen-3-yl]phenyl]5-hydroxypentanoate (Il)

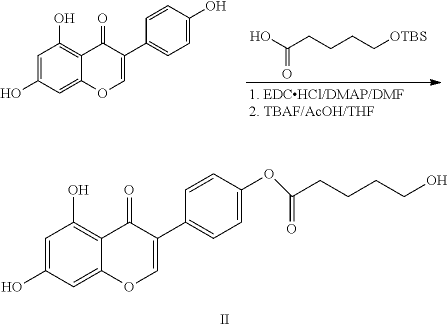 [SMILES: O=c1c(-c2ccc(O)cc2)coc2cc(O)cc(O)c12 ; CC(C)(C)[Si](C)(C)OCCCCC(=O)O ; O=C(CCCCO)Oc1ccc(-c2coc3cc(O)cc(O)c3c2=O)cc1 ; "II"]

Genistein was acylated with 5-(tert-butyldimethylsilyloxy)pentanoic acid in the presence of 1-ethyl-3-(3-dimethylaminopropyl)carbodiimide hydrochloride and 4-dimethylaminopyridine in dimethylformamide to give a mixture consisting primarily of the 4′-ester. Following purification, the hydroxy protecting group on the pentanoate was removed by hydrolysis with tetra-n-butylammonium fluoride and acetic acid in tetrahydrofuran to give Il having 95% purity.

The tri(3-methylbutanoate) derivative Ic was prepared in the same manner as Ib, but using 3-methylbutanoyl 3-methylbutanoate; the di(3-methylbutanoate) derivative Id and the di(acetate) derivative If were prepared in the same manner as Ie, but using 3-methylbutanoyl 3-methylbutanoate and acetic anhydride, respectively. Mixed derivatives such as Ig and Ih are prepared by the mono-acylation of genistein at the 4′-position with a single equivalent of the appropriate acid anhydride, e.g., acetic anhydride for Ig, followed by a further acylation at the 7-position, again using a single equivalent of the appropriate acid anhydride or acyl halide, such as 2-acetoxyacetyl chloride. The phosphonate derivative Ia is prepared using genistein and phosphonyl halide, such as phosphonyl chloride with a base, such as DMAP, Et₃N or pyridine, in an inert solvent such as DCM. Amino acid derivatives such as Ii, Ij, and Ik may be prepared by the mono-acylation of genistein at the 4′-position with a single equivalent of the amino acid or an activated derivative, with the amine group protected in the conventional manner, and then deprotected. Derivatives at other positions are prepared by, for example, protection of the genistein 4′-hydroxy group by alkylation, acylation of the protected genistein, and deprotection.

### EXAMPLES

Details of these examples, other than Example 9, are described in full in Wei et al., “Cannabinoid receptor 1 antagonist genistein attenuates marijuana-induced vascular inflammation”, Cell, 185(10), 1676-1693 (2022), published online on 29 Apr. 2022: https://doi.org/10.1016/j.cell.2022.04.005, and the supplemental materials to that article, which are incorporated herein by reference.

### Example 1: Biobank Screening for Association Between Cannabis Use and Adverse Cardiovascular Events

The UK Biobank is a large prospective cohort study, containing genetic and phenotypic data on 500,000 individuals aged 40-69, recruited between 2006 and 2010 and serially followed over time (sec Bycroft et al., “The UK biobank resource with deep phenotyping and genomic data”, Nature, 562, 203-209 (2018)). The number of individuals in the UK Biobank who were cannabis users versus non-cannabis users (http://biobank.ctsu.ox.ac.uk/crystal/field.cgi?id=20453). Individuals who responded “no” to having ever used cannabis were identified as non-cannabis users; while the remaining individuals were segregated based on cannabis frequency use, and individuals who responded “yes” to cannabis use more than once/month were identified as cannabis users. The number of cannabis users and non-cannabis users reported as having a previous episode of myocardial infarction (MI) was obtained; and the incidence of MI in cannabis and non-cannabis users was then determined. To determine the incidence of MI in cannabis and non-cannabis users under the age of 50, the same procedure was followed for cannabis and non-cannabis users under the age of 50 at the time of the survey. Of 157,331 Biobank participants who had completed the cannabis use survey, 122,455 were identified as non-cannabis users, in whom 2776 MIs were noted, with 548 premature MIs (MIs under the age of 50), for an incidence of 0.45%; while 11,914 were identified as cannabis users, in whom 217 MIs were noted, with 63 premature MIs, for an incidence of 0.53%. When controlling for age, body mass index, and sex, cannabis use was a statistically significant positive predictor for MI using a logistic regression model.

Inflammation is central in the pathogenesis of atherosclerosis, and markers of inflammation using an Olink proteomic analysis of plasma from recreational smokers were examined. Twenty recreational marijuana smokers, who had abstained from using marijuana for 24 hr, were previously recruited to smoke a single marijuana cigarette and had serial blood draws to assess for Δ⁹-THC levels in plasma (Lynch et al., “Correlation of breath and blood A9-tetrahydrocannabinol concentrations and release kinetics following controlled administration of smoked cannabis”, Clin. Chem., 65, 1171-1179 (2019). After smoking a marijuana cigarette, plasma samples for 18 participants were serially drawn using sodium citrate tubes at 15 min intervals over 180 minutes. The Olink inflammation panel analysis revealed that several inflammatory cytokines were upregulated at 90 minutes including: TGFβ, CCL4, CCL19, CXCL6, CXCL10, CXCL11, IL8, MCP2, MCP4, TNF, and CCL1. These upregulated cytokines are all implicated in atherosclerosis. FLT3 is downregulated after marijuana smoking, which is also associated with accelerated atherosclerosis in Flt3^{(-/-)}Ldlr⁽⁻¹⁻⁾ mice.

### Example 2: Genistein Binds to the CB1 Receptor and Inhibits CB1 Activity

Genistein was identified as a potential CB1 antagonist by virtual screening the SWEETLEAD chemical database against four known selective CB1 antagonists (rimonabant, otenabant, AM251, and DBPR-211) using the ROCS software suite, and molecular docking with the Schrodinger GLIDE docking functionality and the selective CB1 antagonist AM6538 as positive control. An in vitro GTPase assay found that genistein functions as a neutral antagonist against CB1, and radioligand binding assays for predicted targets of genistein binding revealed that genistein binds human CB1 with an IC₅₀ of 150 nM.

### Example 3. Δ⁹-THC Induces Cytotoxicity in Human Endothelial Cells

Δ⁹-THC cytotoxicity was examined in three different cell types: human endothelial cells (human umbilical vein endothelial cells (HUVECs) and human coronary artery endothelial cells (HCAECs)), human embryonic stem cell-derived cardiomyocytes (H7 hESC-CMs), and human cardiac fibroblasts-ventricular (NHCF-V). Δ⁹-THC induced cytotoxicity in human endothelial cells, without any adverse effect on cardiomyocytes or cardiac fibroblasts. Inflammation and oxidative stress cause endothelial dysfunction. Δ⁹-THC induced the expression of inflammation-related genes and reduced the antioxidant-related gene expression. Because HUVECs were derived from large pools of female patients with environmental exposures that might affect the phenotype with Δ⁹-THC, while human induced pluripotent stem cell-derived endothelial cells (hiPSC-ECs) are free of previous environmental exposures, hiPSC-EC lines were generated from healthy individuals. hiPSC-ECs formed capillary-like tubes mimicking angiogenesis in vitro and Δ⁹-THC also induced cytotoxicity. The expression of cannabinoid receptors in hiPSC-ECs using hiPSC-derived neurons and human erythroblast cells (HEL92.1.7) as positive controls was examined. The CB1 receptor, but not the CB2 receptor, was expressed in hiPSC-ECs, thus hiPSC-ECs can be used to model the effects of Δ⁹-THC on the vasculature via the CB1 receptor.

Δ⁹-THC also decreased mRNA expression of antioxidant-related genes including superoxide dismutase 1 (SOD1), superoxide dismutase 2 (SOD2), catalase (CAT), and glutathione peroxidase 1 (GPX1). In addition, the ROS-related genes implicated in endothelial dysfunction such as NADPH oxidase 1 (NOX1) and inducible nitric oxide synthase (iNOS) were upregulated in hiPSC-ECs after Δ⁹-THC treatment. Immunofluorescence staining showed that Δ⁹-THC treatment of hiPSC-ECs also induced cellular oxidative stress with increased hydrogen peroxide (H₂O₂) levels.

### Example 4: Δ⁹-THC Induces Inflammation and NF-κB Activation in hiPSC-ECs

The expression of inflammation-related genes in hiPSC-ECs after treatment with Δ⁹-THC was examined. Proinflammatory cytokines and chemokines were increased in response to Δ⁹-THC, while I kappa B (IκB), a specific inhibitor of the NF-κB transcription factor, was inhibited by Δ⁹-HC. TNFα contributes to vascular dysfunction; and, after Δ⁹-THC treatment, the TNFα concentration was increased in the cell culture medium of hiPSC-ECs. Monocyte adhesion to endothelial cells is associated with the development of atherosclerosis; and the adhesion of monocytes to hiPSC-ECs after Δ⁹-THC treatment suggested that monocytes have increased adhesion to hiPSC-ECs. The longitudinal effects of Δ⁹-THC-induced inflammation were assessed in hiPSC-ECs using wash-out experiments. After treatment with Δ⁹-THC for 48 hours, the supernatant in hiPSC-ECs was replaced with fresh medium. The expression of inflammation-related genes in hiPSC-ECs was evaluated every other day for two weeks. The Δ⁹-THC-induced expressions of proinflammatory cytokines and chemokines was sustained for 8 to 10 days after the initial exposure. Δ⁹-THC induced inflammation persists four to five times longer than the initial exposure.

### Example 5: Genistein reverses Δ⁹-THC-induced oxidative stress and inflammation in hiPSC-ECs by inhibiting CB1

The CB1 receptor is implicated in the pathological effects of Δ⁹-THC on the vasculature. Pharmacologic and genetic inactivation of CB1 receptor may be used to determine whether the CB1 receptor plays a role in Δ⁹-THC-induced effects in hiPSC-ECs. The selective CB1 antagonist AM6545 reversed Δ⁹-THC-induced mRNA expression of inflammation- and oxidative stress-related genes; and the Δ⁹-THC-induced effects were also reversed by the knockdown of CB1 by siRNA. CRISPR interference (CRISPRi) of CB1 expression reversed Δ⁹-THC-induced inflammation and oxidative stress in hiPSC-ECs. Genistein is known to ameliorate TNFα mediated inflammation and oxidative stress in endothelial cells (Jia et al., “Genistein inhibits TNF-alpha-induced endothelial inflammation through the protein kinase pathway A and improves vascular inflammation in C57BL/6 mice”, Int. J. Cardiol., 168, 2637-2645 (2013)); thus, the CB1 receptor is required for genistein to attenuate the inflammatory effects of TNFα.

In hiPSC-ECs, genistein ameliorated the expression profile of antioxidant genes that are suppressed by Δ⁹-THC, while also having a salutatory effect on iNOS and NOX1 expression. hiPSC-ECs treated with genistein and Δ⁹-THC are more likely to remain quiescent and not contribute to the pathogenesis of atherosclerosis. Genistein not only reversed Δ⁹-THC-induced monocyte adhesion to hiPSC-ECs but also attenuated NF-κB phosphorylation.

Toll-like receptors (TLRs) are components of the innate immune system that recognize molecular patterns of microbial components and TLR4/NF-κB signaling pathway can contribute to vascular inflammation in endothelial cells. Δ⁹-THC induced TLR4 expression in hiPSC-ECs, whereas genistein disrupted its expression. And genistein reversed the Δ⁹-THC-induced mRNA expression of proinflammatory cytokines and chemokines. Genistein also shortened the recovery time of Δ⁹-THC-induced inflammation from 8-10 days to 4-6 days. Genistein attenuates Δ⁹-THC-induced oxidative stress and inflammation in hiPSC-ECs.

### Example 6: Genistein reverses Δ⁹-THC-induced endothelial dysfunction, oxidative stress, and inflammation in a mouse model

Male C57BL/6J mice were treated with intraperitoneal injection of vehicle, Δ⁹-THC, or Δ⁹-THC plus genistein. While oral ingestion or smoking are more common routes of ingestion in humans, this mouse model produces a plasma concentration of Δ⁹-THC of ˜100 ng/ml as revealed by LC-MS analysis, which is comparable to the concentration from smoking a single marijuana cigarette. Seven-week-old male C57BL/6J mice were obtained from Jackson Laboratory (Bar Harbor, ME, USA). The mice were treated with vehicle, Δ⁹-THC, or Δ⁹-THC plus genistein (n=5/group) for 30 days and then sacrificed. Organs were collected, weighed, and fixed with 4% paraformaldehyde (PFA). The thoracic aortas (TAs) were surgically dissected out and cleaned from the surrounding connective tissue for all groups. The TAs were transferred to ice-cold Krebs'-Henseleit solution and cut into segments of 2 mm. The artery segments were mounted in wire-myograph chambers for isometric tension recordings (DanishMyo Technology [DMT]).The chambers contained Krebs'-Henseleit solution. All chambers were aerated with 95% 02/5% CO2, and the temperature was kept constant at 37° C. to mimic physiological conditions. After a manual stabilization and normalization procedure, the PowerLab4/25-Chart7 acquisition systems (AD Instruments Ltd) was used to record force and convert it into tension. The TA segments were pre-constricted using either 1-3 mM 5HT serotonin (5HT) or U46619. A cumulative application of acetylcholine (1-10 mM) was performed, and the induced vascular dilatations were measured. Subsequently, concentration-response relationship curves were established for each group of 3 mice. Time-matched dimethylsulfoxide (DMSO) application was performed in a separate chamber as the control for all experiments. Wire myograph reveals that Δ⁹-THC induced endothelial dysfunction in mice, whereas genistein mitigated the effect.

Genistein and the known CB1 antagonist rimonabant ameliorated mRNA expressions of inflammation induced by Δ⁹-THC in mouse thoracic artery tissues; and genistein also rescued the oxidative stress-related gene expression. Expression of inflammation and oxidative stress-related genes correlates with vascular relaxation. NF-κB phosphorylation and SOD expression in mouse serum were also attenuated with genistein. Δ⁹-THC administration decreased circulating levels of the antioxidant, glutathione, which was ameliorated with genistein co-treatment. Analysis of C57BL/6J plasma reveals that interleukins IL-6, IL-3 and IL-10, which are associated with an increased risk of atherosclerosis, were elevated after treatment with Δ⁹-THC, and genistein cotreatment significantly reduced the expression of these inflammatory cytokines. Collectively, these results indicated that genistein can reverse Δ⁹-THC-induced effects in vivo. While genistein had a protective effect on the vasculature, it did not exhibit any toxicity based on body weight and H&E staining of various organs.

### Example 7: Effects of Δ⁹-THC and Genistein on Atherosclerosis

To investigate the effect of Δ⁹-THC and genistein in chronic atherosclerosis formation, LDL receptor knock-out mice (B6.129S7-Ldlr^{tm1Her/J}) were fed a high-fat diet for 12 weeks and treated with: vehicle control, Δ⁹-THC, or Δ⁹-THC and genistein (n=10-12/group). All groups showed an increase in body weight, and the successful administration of Δ⁹-THC was confirmed in serum by LC-MS at 12 weeks. Neither Δ⁹-THC nor cotreatment with Δ⁹-THC and genistein affected serum lipid profiles or blood pressure. After 12 weeks, the mice were euthanized, and the atherosclerotic lesion area was examined by cross-sections of the aortic root and an en face analysis of the thoracic aorta. In both the cross-sectional analysis and en face analysis, when stained with Oil Red O, mice treated with Δ⁹-THC showed significantly increased plaque size, and co-treatment with genistein ameliorated plaque size. Macrophage recruitment in the aortic root was analyzed using aortic plaque stained with an anti-CD68 antibody. The CD68-positive area was significantly increased by Δ⁹-THC administration and rescued by co-treatment with genistein. These results suggest that Δ⁹-THC exacerbated atherosclerosis formation and macrophage recruitment in atherosclerotic plaques, which were ameliorated by genistein co-treatment.

An ApoE^{-/-} mouse model was also employed to investigate the effects of Δ⁹-THC and cotreatment with genistein on atherosclerosis. Partial carotid artery ligation (PCAL) was performed in ApoE^{-/-} mice at 10-16 weeks. After PCAL, the mice were divided into three groups: vehicle control; Δ⁹-THC; Δ⁹-THC plus genistein (n=5/group). All mice were fed a high-fat diet for 10 days. At the end of the treatment period, the mice were euthanized and subjected to histological analysis. The ligated carotid artery showed increased fat deposition with Oil Red 0 staining compared to the unligated, contralateral vessel; and H&E staining suggested neointimal thickening, fat deposition, and increased macrophage recruitment in ligated carotid atherosclerotic plaques. Oil Red 0 staining found that plaque area was increased by Δ⁹-THC treatment, whereas Δ⁹-THC and genistein cotreatment ameliorated plaque size. Macrophage recruitment was tested using the macrophage-specific F4/80 antibody and CD68 antibody. In the ligated carotid artery, the F4/80-positive area was significantly increased by Δ⁹-THC administration and decreased by co-treatment with genistein. Immunohistochemical analysis with CD68 specific antibody revealed the CD68-positive area was significantly increased by Δ⁹-THC administration and reduced by co-treatment with genistein. Consistent with the Ldlr^{-/-} model findings, the ApoE^{-/-} PCAL mouse model found increased plaque size and reduced macrophage recruitment with Δ⁹-THC, and genistein cotreatment ameliorated these indices of atherosclerosis in ligated carotid arteries.

### Example 8: Genistein is Predominantly Peripherally Restricted, Binds the CB1 Receptor and does not Attenuate Δ⁹-THC Neurobehavioral Effects In Vivo

Although genistein attenuated the vascular dysfunction caused by Δ⁹-THC in both in vitro and in vivo studies, direct evidence of genistein binding CB1 was explored with a fluorescently-labelled genistein (BODIPY517/547-genistein). In CB1 radioligand binding assays, BODIPY517/547-genistein has an IC₅₀ of 357 nM and labels nearly all hiPSC-ECs cells after a 12 hour incubation. In vivo binding is investigated using intravenously injected BODIPY517/547-genistein in C57BL/6J mice. After 48 hr, BODIPY517/547-genistein is detected in the abdominal viscera, thoracic aorta, heart, and lungs, but is minimally detected in the brain. This is consistent with previous report (Yang et al., “Bioavailability and pharmacokinetics of genistein: mechanistic studies on its ADME”, Anticancer Agents Med. Chem., 12, 1264-1280 (2012)) showing genistein has poor blood-brain barrier (BBB) penetration.

Neurobehavioral testing (Billy Martin tetrad): Despite BODIPY517/547 having excellent bioavailability, genistein had poor BBB penetration, and it was therefore worthwhile to interrogate the neurological effects of Δ⁹-THC in the context of genistein co-administration. The neurobehavioral effects of Δ⁹-THC include decreased mobility, analgesia, hypothermia, and sedation, which are described as the Billy Martin tetrad (see, e.g., Wiley et al., “Cannabinoid pharmacological properties common to other centrally acting drugs”, Eur. J. Pharmacol., 471, 185-193 (2003)). The sedative and analgesic effects of Δ⁹-THC may have therapeutic benefit; and, while protecting against the detrimental vascular effects, genistein may antagonize the neurobehavioral tetrad effects of Δ⁹-THC. Therefore, the neurological effects of Δ⁹-THC and genistein co-administration were tested in C57BL/6J mice treated with vehicle, genistein, Δ⁹-THC, or in combination. The tetrad effects were assessed using an activity chamber for mobility, a hot plate for analgesia, a rectal probe for hypothermia, and the bar test for sedation. Group housed, 6-7 weeks old, male C57B1/6J mice from Jackson Laboratory were used in the study. All behavioral tests were conducted during the animals' dark cycle. The mice were randomized into different dosing groups based on their body weight. Δ⁹-THC (20 mg/kg i.p., in a vehicle of 5% ethanol, 5% Cremophor EL, and 90% isotonic saline) and genistein (50 mg/kg p.o., in a corn oil vehicle) were used in the study, with groups as follows: Group 1: n=8, Veh i.p.+Veh p.o.; Group 2: n=9 THC i.p.+Veh p.o.; Group 3: n=8 Veh i.p.+genistein p.o.; Group 4: n=9 THC i.p.+genistein p.o.). Δ⁹-THC caused decreased mobility (open field activity chamber; tested pre-dosing and 60 min post-dosing), analgesia (hot plate apparatus set to 55° C.; 30 sec maximum trial duration; latency to hind paw licking, flicking, or jumping was recorded), hypothermia (rectal body temperature; tested pre-dosing and 60 min post-dosing), and sedation (bar test; a small metal rod (0.7 cm diameter, 10 cm long) was suspended 3-5 cm above the table and the forepaws of the mouse were placed on the bar. The total duration the mice spent with one or both paws on the bar in a catalepsy state is recorded for a total of 60 sec. If the mouse jumped over the bar, it was placed in the original position on the bar a maximum of 4 times. A score of 0 seconds is assigned to a mouse if it jumped over the bar a fifth time; tested pre-dosing and 60 min post-dosing) in C57BL/6J mice. Genistein alone did not affect tetrad effects, and genistein could not attenuate the Δ⁹-THC neurobehavioral effects in co-treatment.

### Example 9: Human Studies with Genistein and Genistein Derivatives

Human subjects ingesting medicinal cannabinoids, such as dronabinol and nabilone, are randomized to treatment with placebo or with genistein at 1 or 5 mg/Kg/day. Subjects treated with genistein are found to exhibit a dose-related lower incidence of adverse cardiovascular effects of their cannabinoid ingestion. A similar study with a genistein derivative yields similar results.

While this invention has been described in conjunction with specific embodiments and examples, it will be apparent to a person of ordinary skill in the art, having regard to that skill and this disclosure, that equivalents of the specifically disclosed materials and methods will also be applicable to this invention; and such equivalents are intended to be included within the following claims.

What is claimed is:

1. A method of treating an adverse cardiovascular effect of a cannabinoid in a subject, comprising administration to the subject of a therapeutically effective amount of genistein or a genistein derivative.

2. The method of claim 1, comprising administration to the subject of a therapeutically effective amount of a genistein derivative.

3. The method of claim 1 or 2 where the genistein or genistein derivative is a compound of formula I: 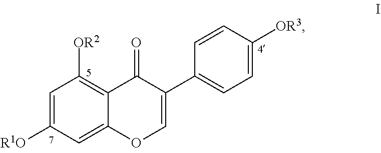
or a pharmaceutically acceptable salt thereof,
where each of R¹, R² and R³ independently is selected from the group consisting of H, (HO)₂P(O)—, HOS(O)₂—, H₂NS(O)₂—, H(OCH₂CH₂)₁₋₁₀—, (CH₃)₂NCH₂CH₂—, morpholino, (C₁₋₆ alkyl)C(O)— [where the C₁₋₆ alkyl is optionally substituted with 1 or 2 substituents selected from the group consisting of halo, hydroxy, C₁₋₄ alkoxy, amino, C₁₋₄ alkylamino, cyano, and acetoxy], and an acyl group of an amino acid or hydroxy acid.

4. The method of claim 3, where the C₁₋₆ alkyl is selected from the group consisting of methyl, ethyl, isopropyl, isobutyl; and (acetyloxy)methyl, (1-acetyloxy)ethyl or (2-acetyloxy)ethyl

5. The method of claim 3 where the amino acid is alanine, aspartic acid, cysteine, glutamic acid, glycine, valine, isoleucine, leucine, methionine, phenylalanine, proline, serine, threonine, tyrosine or tryptophan.

6. The method of claim 3 where the hydroxy acid is an ω-hydroxy acid.

7. The method of claim 6 where the hydroxy acid is δ-hydroxypentanoic acid.

8. The method of claim 3 where the genistein derivative is a compound of formula Ia to Il: 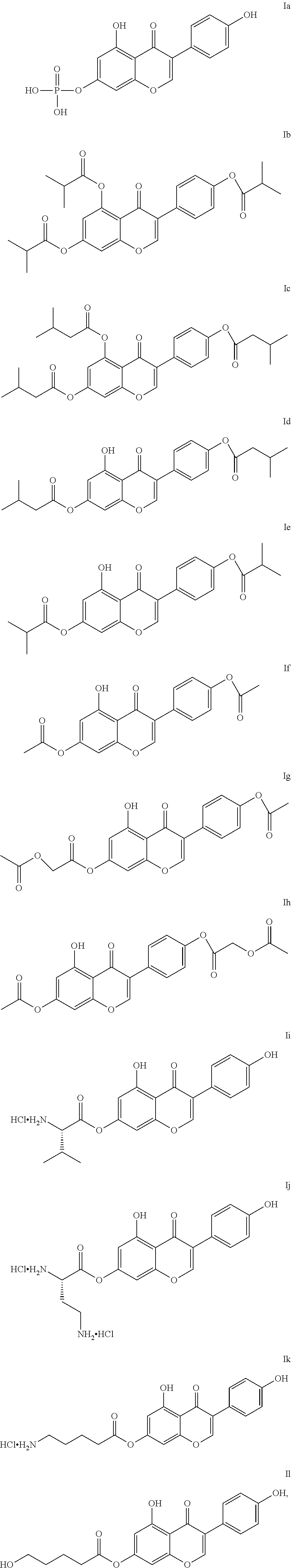
or a pharmaceutically acceptable salt thereof.

9. The method of claim 1, where the genistein or a genistein derivative is formulated as a nanoparticulate suspension.

10. The method of claim 9 where the genistein or a genistein derivative consists of particles have a median particle size of less than about 200 nm, less than 150 nm, less than 100 nm, less than about 90 nm, less than about 80 nm, less than about 70 nm or less than about 60 nm.

11. The method of claim 1 where the genistein or a genistein derivative is formulated with a water soluble polymer.

12. The method of claim 1 where the dose of the genistein or a genistein derivative is 1-5 mg/Kg/day.

13. The method of claim 1 where the cannabinoid is selected from the group consisting of Δ⁹-tetrahydrocannabinol, nabilone, or cannabidiol, or a mixture thereof.

14. The method of claim 1, wherein the adverse cardiovascular effect comprises one or more of: upregulation of pro-inflammatory cytokines and chemokines, monocyte adhesion to cardiovascular endothelial cells, oxidative stress-related gene expression such as NF-kB phosphorylation and SOD expression in endothelial cells.

15. The method of claim 1, wherein the adverse cardiovascular effect is selected from the group consisting of myocardial infarction (MI), angina, arrhythmias, endothelial dysfunction, atherosclerosis, cardiomyopathy and metabolic dysfunction.
